(19) United States
(12) Patent Application Publication
(10) Pub. No.: US 2010/0221398 A1
(43) Pub. Date: Sep. 2, 2010

(54) PRESERVATIVE

(76) Inventors: David Brian Archer, Nottingham (GB); Malcolm Stratford, Nottingham (GB); Andrew Plumridge, Nottingham (GB)

Correspondence Address: NIXON & VANDERHYE, PC, 901 NORTH GLEBE ROAD, 11TH FLOOR, ARLINGTON, VA, 22203, US

(21) Appl. No.: 12/601,056

(22) PCT Filed: Jun. 6, 2008
(86) PCT No.: PCT/GB2008/001935

§ 371 Date: May 7, 2010

(30) Foreign Application Priority Data
Jun. 7, 2007 (GB) 0710884.8

(51) Int. Cl. A23L 3/3499; C07C 57/10; C07C 63/64; C09K 15/06

(52) U.S. Cl. 426/335; 562/601; 562/495; 252/407

(57) ABSTRACT

The invention relates to use of a compound of Formula (I) as a preservative, or to enhance the anti-mould efficacy of another preservative and a product comprising a compound of Formula (I), wherein: the second carbon is optionally substituted; the bond between R and the second carbon is unsaturated; R is a C₁ to C₂₀ alkyl, aryl, alkaryl, alkenyl or alkynyl; and wherein R may be optionally substituted; Z is H or OH; and when Z is OH, the bond between R and the second carbon is a triple bond.

The invention relates to compounds for use as food preservatives, and in particular as preservatives to reduce or prevent mould growth in or on foods.

Fungal spoilage represents a major problem to the food industry. The nutrients present in food for human or animal consumption often form an ideal substrate for fungal proliferation. Foods where fungal growth exceeds 10⁵ cells/ml (or equivalent biomass) are usually regarded as spoiled. The total cost of fungal spoilage of foods worldwide is likely to exceed $1 billion/annum, with up to a quarter of all foods lost to spoilage. Fungal spoilage can be caused by yeasts or moulds, in each case the symptoms of spoilage are different. For example, yeasts characteristically ferment sugars in foods, causing the packaging to “blow” and bottles to explode due to gas formation, whereas moulds cause the formation of particulates, films, surface colonies or submerged hyphae, as well as causing alterations in food taste and odour due to the formation of secondary metabolites. Growth of moulds on food, either for human or animal consumption, can form a threat to health through the formation of a variety of mycotoxins.

Mould contamination of food can occur predominantly in one of three forms: (i) vegetative hyphal filaments; (ii) conidiospores (vegetative spores designed for aerial dispersal); and (iii) sexual spores (ascospores or basidiospores). The most probable source of mould infection into foods is via the raw materials used or via aerial infection. Infected raw materials, such as damaged fruit or mouldy cereal grains, may contain hyphae, conidiospores and small numbers of sexual spores, while aerial infection is almost exclusively due to conidiospores.

Fungi are able to proliferate in a wide range of environments. Many fungi are able to grow at a wide pH range, pH 1.5-pH 10.0, across a temperature range 0-40° C., and in foods dehydrated by salt or sugar to a water activity of 0.61, but are sensitive to heat above 70° C. Since most uncooked foods fall within this environmental range, it is clear that many foods are possible targets for fungal spoilage. In practice, mould spoilage is most prevalent in low pH foods or beverages or dehydrated foods such as raisins or cereal grains. The genera of mould most commonly encountered in food spoilage flora are Penicillium, Trichoderma and Aspergillus. Fungal food spoilage is reviewed by Pitt and Hocking (1997) in Fungi and Food Spoilage 2^{nd} Edn., Blackie Academic and Professional: London, Weinheim, New York, Melbourne, Madras.

In addition to spoilage by fungi, food is also vulnerable to spoilage by other microbes such as bacteria. To try to prevent and/or reduce spoilage by microbes, preservatives are routinely added to foods.

Any preservative for food use must fulfil the following criteria: (i) be proven safe for human and/or animal consumption; (ii) be legally permitted within the country of use; (iii) have antimicrobial activity in the conditions of the food and against the expected spoilage flora; and (iv) have a limited impact on the flavour and taste of the food. Very few chemical agents fulfil this stringent specification. To prevent fungal (and bacterial) spoilage of foods, a limited range of chemical additives have been approved for use as food preservatives in the E.U. These include sorbic acid, benzoic acid, acetic acid and propionic acid, and in certain foods the addition of sulphites and nitrites are permitted.

Sorbic acid, otherwise known as 2,4-hexadienoic acid, is a widely-used food preservative that is particularly effective in acidic conditions. Foods preserved with sorbic acid typically have a low pH and contain sugar, such as soft drinks and fruit juices, jams and confectionary. Sorbic acid is particularly effective against bacteria, almost all yeast species, but only some moulds. Cinnamic acid is a recognised food flavouring agent used in baked goods, frozen dairy, soft candy and beverages, which also has considerable antimicrobial effect against bacteria and spoilage yeasts, but, like sorbic acid, is largely ineffective against moulds. Cinnamic acid is a phenylacrylic acid, which naturally occurs in cinnamon, basil and balsam (Burdock (1995) Fenarli's Handbook of Flavor Ingredients Volume II 3^{rd} Edn., CRC Press: Boca Raton, Ann Arbour, London, Tokyo).

Many moulds are capable of causing spoilage in foods preserved with sorbic acid if the mould inoculum is sufficiently high. Plumridge et al. (2004) in Applied and Environmental Microbiology 70, 3506-3511 found a strong inoculum effect using Aspergillus niger conidiospores in media containing sorbic acid. That is, high concentrations of spores required considerably greater concentrations of sorbic acid in order to prevent mould growth.

The explanation for this is believed to lie in the ability of A. niger conidiospores to degrade sorbic acid (and similar acids, such as cinnamic acid) thereby inactivating the preservative/anti-microbial effect of the acid. After an initial delay of a few hours, germinating conidiospores will degrade all detectable sorbic acid over the course of 18 hours, before completing their outgrowth and rapidly proliferating in the now sorbic-acid free medium (Plumridge et al. (2004) Applied and Environmental Microbiology 70, 3506-3511). Experiments have shown that in the presence of the conidiospores of A. niger, sorbic acid is decarboxylated to produce the volatile hydrocarbon, 1,3-pentadiene.

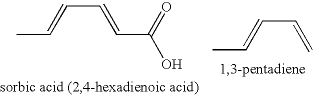

Resistance of spoilage moulds to sorbic acid has been observed primarily in the non-sexual conidiospores of moulds. Mould hyphae (from 24-hour germinated spores) have been shown to have very low sorbic acid degrading activity, and consequently are very sensitive to inhibition by sorbic acid. In addition to degrading sorbic acid, studies have shown that germinating conidiospores also have the ability to completely degrade cinnamic acid to styrene. The ability of the conidiospores to cause degradation of preservatives, such as sorbic acid and cinnamic acid, has been shown in several mould genera, and is particularly strong in the most preservative-resistant spoilage species, including Aspergillus, Trichoderma and Penicillium spp.

The protein believed to be responsible for degradation of preservatives has been identified as PadA1 in A. niger, which is encoded by the gene padA1. The Pad1p protein, encoded by PAD1, has been previously reported to cause degradation of cinnamic acid to styrene in the yeast Saccharomyces cerevisiae, (Pad=Phenyl Acrylic acid Decarboxylase). PadA1 and Pad1p are also able to decarboxylate many other acids including sorbic acid. Table 1 lists acids that are decarboxylated by PadA1 in A. niger conidiospores, and the product produced by the decarboxylation.

TABLE 1
PadA1 substrates and products

| Substrate | MW | Product | MW |
|---|---|---|---|
| Sorbic acid | 112.128 | 1,3-Pentadiene | 68.1182 |
| 2,4-Pentadienoic acid | 98.1012 | 1,3-Butadiene | 54.0914 |
| 2,4-Heptadieneoic acid | 126.1548 | 1,3-Hexadiene | 82.145 |
| 2,4-Octadienoic acid | 140.1816 | 1,3-Heptadiene | 96.172 |
| 2,4-Nonadienoic acid | 154.2084 | 1,3-Octadiene | 110.199 |
| 2,4-Decadienoic acid | 168.2352 | 1,3-Nonadiene | 124.225 |
| Cinnamic acid | 148.161 | Styrene | 104.1512 |
| 2-Methyl-cinnamic acid | 162.1878 | 2-Methyl-styrene | 118.178 |
| 3-Methyl-cinnamic acid | 162.1878 | 3-Methyl-styrene | 118.178 |
| 4-Methyl-cinnamic acid | 162.1878 | 4-Methyl-styrene | 118.178 |
| 2-Fluoro-cinnamic acid | 166.1515 | 2-Fluoro-styrene | 122.1417 |
| 3-Fluoro-cinnamic acid | 166.1515 | 3-Fluoro-styrene | 122.1417 |
| 4-Fluoro-cinnamic acid | 166.1515 | 4-Fluoro-styrene | 122.1417 |
| 2,4-Difluoro-cinnamic acid | 184.142 | 2,4-Difluoro-styrene | 140.1322 |
| 2,5-Difluoro-cinnamic acid | 184.142 | 2,5-Difluoro-styrene | 140.1322 |
| 2,6-Difluoro-cinnamic acid | 184.142 | 2,6-Difluoro-styrene | 140.1322 |
| 3,4-Difluoro-cinnamic acid | 184.142 | 3,4-Difluoro-styrene | 140.1322 |
| 3,5-Difluoro-cinnamic acid | 184.142 | 3,5-Difluoro-styrene | 140.1322 |
| 2,3,4-Trifluoro-cinnamic acid | 202.1325 | 2,3,4-Trifluoro-styrene | 158.1227 |
| 2,3,4,5,6-Pentafluoro-cinnamic acid | 238.1135 | 2,3,4,5,6-Pentafluorostyrene | 194.1037 |
| 2-Chloro-cinnamic acid | 182.6061 | 2-Chlorostyrene | 138.5963 |
| 3-Choro-cinnamic acid | 182.6061 | 3-Chloro-styrene | 138.5963 |
| 4-Chloro-cinnamic acid | 182.6061 | 4-Chlorostyrene | 138.5963 |
| 2,4-Dichloro-cinnamic aid | 217.0512 | 2,4-Dichlorostyrene | 173.0414 |
| 2-Chloro, 6-fluoro-cinnamic acid | 200.5966 | 2-Chloro, 6-fluoro-styrene | 156.5868 |
| 2-Bromo-cinnamic acid | 227.0571 | 2-Bromo-styrene | 183.0473 |
| 3-Bromo-cinnamic acid | 227.0571 | 3-Bromo-styrene | 183.0473 |
| 4-Bromo-cinnmaic acid | 227.0571 | 4-Bromo-styrene | 183.0473 |
| 2-Trifluoromethyl-cinnamic acid | 216.1593 | 2-Trifluoromethyl-styrene | 172.1495 |
| 3-Trifluoromethyl-cinnamic acid | 216.1593 | 3-Trifluoromethyl-styrene | 172.1495 |
| 4-Trifluoromethyl-cinnamic acid | 216.1593 | 4-Trifluoromethyl-styrene | 172.1495 |
| 2-Methoxy-cinnamic acid | 178.1872 | 2-Methoxy-styrene | 134.1774 |
| 3-Methoxy-cinnamic acid | 178.1872 | 3-Methoxy-styrene | 134.1774 |
| 4-Methoxy-cinnamic acid | 178.1872 | 4-Methoxy-styrene | 134.1774 |
| 3-Ethoxy-cinnamic acid | 192.214 | 3-Ethoxy-styrene | 148.2042 |
| 4-Ethoxy-cinnamic acid | 192.214 | 4-Ethoxy-styrene | 148.2042 |
| alpha-fluoro-cinnamic acid | 166.1515 | (2-fluorovinyl)benzene | 122.1417 |
| alpha-methyl-cinnamic acid | 162.1878 | Propenyl-benzene | 118.178 |
| Furylacrylic acid | 138.1226 | 2-Vinyl furan | 94.1128 |
| 3-Furan-acrylic acid | 138.1226 | 3-Vinyl furan | 94.1128 |
| 3-(2-Thienyl)acrylic acid | 154.1832 | 2-Vinyl thiophene | 110.1734 |
| 3-(3-Thienyl)acrylic acid | 154.1832 | 3-Vinyl thiophene | 110.1734 |

In A. niger, PadA1 activity is not constitutive, but it does appears to be strongly induced in germinating spores by the presence of a substrate acid, for example 1 mM sorbic acid or cinnamic acid.

Deletion of the padA1 gene in A. niger completely abolishes the decarboxylation of sorbic acid and cinnamic acid by mould conidiospores, and renders the conidiospores hypersensitive to inhibition by these acids. The role of padA1 as a resistance gene to sorbic and cinnamic acid is supported by observations that A. niger germlings and hyphae, which show very low levels of PadA1 activity, are also weak-acid sensitive.

An aim of the present invention is to provide a preservative system for use in food which has good anti-microbial activity, in respect of bacteria, yeast and mould.

The present invention provides a compound which can be used as a food preservative, and in particular as a food preservative with anti-mould activity.

Furthermore the present invention provides a preservative system comprising a preservative compound of Formula I (described below) and a second preservative, such as listed as one of the substrates in Table 1, wherein the preservative compound of Formula I prevents or reduces degradation of the second preservative, thereby increasing the anti-mould activity of the second preservative.

According to a first aspect the present invention provides the use of a compound of Formula I as a preservative:

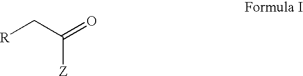

wherein:
the bond between R and the second carbon is unsaturated;
R is a C₁ to C₂₀ alkyl, aryl, alkaryl, alkenyl or alkynyl;

### Z is H or OH; and

when Z is OH, the bond between R and the second carbon is a triple bond.

R may be branched or unbranched. R may be saturated or unsaturated. R may be straight or cyclic. R may be optionally substituted. R may be substituted with OH, ═O, fluoro, chloro, bromo, methyl, ethyl, propyl, butyl etc (a C₁ to C₂₀ alkyl), methoxy, ethoxy, propoxy, butoxy etc (a C₁ to C₂₀ alkoxy) or NR′₂, wherein R′ may be H or C₁ to C₄ alkyl.

The second carbon may be optionally substituted. The second carbon may be substituted with OH, ═O, flouro, chloro, bromo, methyl, ethyl, propyl, butyl etc (a C₁ to C₂₀ alkyl), methoxy, ethoxy, propoxy, butoxy etc (a C₁ to C₂₀ alkoxy) or NR′₂, wherein R′ may be H or C₁ to C₄ alkyl.

Preferably Z is H. Preferably compounds of Formula I are aldehydes.

Preferably the unsaturated bond between the R group and the second carbon is a double or a triple bond.

The R group may be a C₁ to C₁₄ alkyl, aryl, alkaryl, alkenyl or alkynyl.

The R group may be a C₁ to C₉ alkyl, aryl, alkaryl, alkenyl or alkynyl.
The R group may be a C₄ to C₉ alkyl, aryl, alkaryl, alkenyl or alkynyl.
The R group may be a C₄ to C₇ alkyl, aryl, alkaryl, alkenyl or alkynyl.
The R group may be a C₆ to C₉ alkyl, aryl, alkaryl, alkenyl or alkynyl.

Compounds may include at least 4 carbons and may have a double bond at the 2 and 4 positions.

Compounds according to the invention may have an R group which is a C₆ to C₉ alkyl, aryl, alkaryl, alkenyl or alkynyl and a double bond at the 2 and 4 positions.

Preferred examples of compounds of Formula I include 2-pentenal, 2-butenal, 2-hexenal, 2,4-hexadienal, 2-heptenal, 2,4-heptadienal, cinnamaldehyde, 2-octenal, 2,4-octadienal, 2-nonenal, 2,4-nonadienal, phenyl propiolic acid, phenyl propargyl aldehyde, α-amyl-cinnamaldehyde, 2-butyl-2-butenal, α-butyl cinnamaldehyde, citral (Neral, Geranial), 2-ethyl-2-heptenal, 2-furfurylidene butyraldehyde, α-hexyl-cinnamaldehyde, 2-isopropyl-5-methyl-2-hexenal, o-methoxycinnamaldehyde, p-methoxy-α-methylcinnamaldehyde, 3-methyl-2-butenal, α-methylcinnamaldehyde, p-methylcinnamaldehyde, 2-methyl-3(2-furyl)acrolein, 2-methyl-2-octenal, 2-methyl-2-pentenal, 4-methyl-2-pentenal, 5-methyl-2-phenyl-2-hexenal, 4-methyl-2-phenyl-2-pentenal, 2-(methylthio)methyl-2-butenal, 2 -methylthiomethyl-3-phenylpropenal, nona-2-trans,6-cis,dienal, nona-2-trans,6-trans,dienal, 2,6-octadienal, 2,4-pentadienal and 2-phenyl-2-butenal.

The skilled man will appreciate that the preferred features discussed above with reference to Formula I may apply to Formula I as referred to in all aspects on the invention.

The use according to the first aspect of the invention may be as a food preservative, and/or it may be as a non-food preservative, for example as a preservative in home and personal care products such as creams, shampoo and cosmetics.

A preservative, preferably a food preservative, is one or more compounds which act alone or in combination to inhibit, that is, prevent or reduce, microbial deterioration of a product, preferably of a food product. Microbial deterioration may be caused by one or more of bacteria, yeast and mould.

Preferably the use of this aspect of the invention is as a preservative with anti-mould activity. Preferably as a food preservative with anti-mould activity. Preferably the preservative of the invention acts by protecting another preservative from degradation and thus inactivation. Preferably the other preservative is a carboxylic acid. Preferably the carboxylic acid has unsaturated bonds at the second and fourth position. Preferably the unsaturated bond at the second position is a double bond. Preferably the unsaturated bond at the fourth position is a double bond or equivalent unsaturation. Preferably the carboxylic acid is sorbic acid, cinnamic acid, 4-methylcinnamic acid or any of the acids listed as a substrate in Table 1 which also has preservative properties. Most preferably, the carboxylic acid is sorbic acid.

According to another aspect, the invention provides use of a compound of Formula I to enhance the anti-mould efficacy of another preservative, preferably a food preservative.

Preferably a compound of Formula I acts as a preservative protector, and prevents or reduces degradation of another preservative compound.

Preferably the compound of Formula I is used in combination with another preservative to form a more effective preservative system than either compound alone.

Preferably the other preservative is a carboxylic acid. Preferably the carboxylic acid has unsaturated bonds at the second and fourth position. Preferably the unsaturated bond at the second position is a double bond. Preferably the unsaturated bond at the fourth position is a double bond or equivalent unsaturation. Preferably the carboxylic acid is sorbic acid, cinnamic acid, 4-methylcinnamic acid or any of the acids listed as a substrate in Table 1 which has preservative properties. Most preferably, the carboxylic acid is sorbic acid.

Preferably the compound of Formula I is used at food grade purity.

Surprisingly compounds according to Formula I have been shown to be efficacious as food preservatives, in particular when used in combination with known carboxylic acid food preservatives such as sorbic acid. Compounds according to Formula I have been shown to enhance the anti-mould properties of known carboxylic acid food preservatives, such as sorbic acid. Compounds of Formula I are believed to work to enhance the efficacy of known carboxylic acid derivatives by preventing mould from degrading the carboxylic acid and inhibiting its preservative effects.

According to a further aspect the invention provides a preservative composition or system comprising a first preservative and a compound of Formula I. Preferably the first preservative is a carboxylic acid Preferably the preservative composition/system is for use as a food preservative.

Preferably the carboxylic acid has unsaturated bonds at the second and fourth positions. Preferably the unsaturated bond at the second position is a double bond. Preferably the unsaturated bond at the fourth position is a double bond or equivalent unsaturation. Preferably the carboxylic acid is sorbic acid, cinnamic acid, 4-methylcinnamic acid or any of the acids listed as a substrate in Table 1 which has preservative properties. Most preferably, the carboxylic acid is sorbic acid.

Compositions according to any aspect of the invention may also comprise one or more additional agents such as a flavouring agent, a colouring agent, a stabiliser and/or an emulsifier.

According to another aspect the invention provides the use of a composition comprising a carboxylic acid and a compound according to Formula I as a preservative. Preferably the preservative is a food preservative

According to a further aspect, the present invention provides a method of preserving a food comprising adding a compound of Formula I to the food.

A second preservative may also be added to the food. The second preservative may be added before or after or simultaneously to the compound of Formula I. The second preservative may be a carboxylic acid. The carboxylic acid may have unsaturated bonds at the second and fourth positions. Preferably the unsaturated bond at the second position is a double bond. Preferably the unsaturated bond at the fourth position is a double bond or equivalent unsaturation. Preferably the carboxylic acid is sorbic acid, cinnamic acid, 4-methylcinnamic acid or any of the acids listed as a substrate in Table 1 which has preservative properties. Most preferably, the carboxylic acid is sorbic acid.

According to a yet further aspect, the present invention provides a method of preserving food comprising adding a first food preservative and a compound of Formula I to the food.

According to another aspect the invention provides a product, preferably a food product, comprising a compound of Formula I.

According to another aspect the invention provides a product, preferably a food product, comprising a first preservative and a compound of Formula I.

The first preservative may be carboxylic acid. The carboxylic acid may have unsaturated bonds at the second and fourth positions. The unsaturated bond at the second position may be a double bond. The unsaturated bond at the fourth position may be a double bond or equivalent unsaturation. The carboxylic acid may be sorbic acid, cinnamic acid, 4-methylcinnamic acid or any of the acids listed as a substrate in Table 1 which has preservative properties. Preferably, the carboxylic acid is sorbic acid.

The compound of Formula I may be included to enhance the anti-mould activity of the first preservative. The compound of Formula I may reduce or prevent degradation of the first preservative by mould spores in or on the product.

Preferably food, with reference to any aspect of the invention, includes beverages, such as fruit juices and soft drinks, confectionary, perishables such as vegetables, fruit, meat and fish, prepared food typically for sale in tins, pouch or as so called “ready meals” etc and any other food susceptible to bacterial or fungal infection.

The amount of a compound of Formula I and/or a second preservative added to a product, such as a food, will depend on the level of existing microbial contamination, the kind and quality of the product, how the product has been processed, how the product is to be stored and for how long it is to be stored, as well as numerous other factors.

Preferably, a compound of Formula I will be used at about 1 ppm to about 100 ppm in combination with another preservative, such as sorbic acid, which is used at about 200 ppm to about 500 ppm. Preferably, in a food with about 300 ppm of a second preservative, such as sorbic acid, about 5 to about 70 ppm, more preferably about 5 to about 20 ppm, of a compound of Formula I will be used.

The following examples and figures are intended to illustrate the invention. The invention is not limited by the examples and the examples should not be construed to limit the invention in any way.

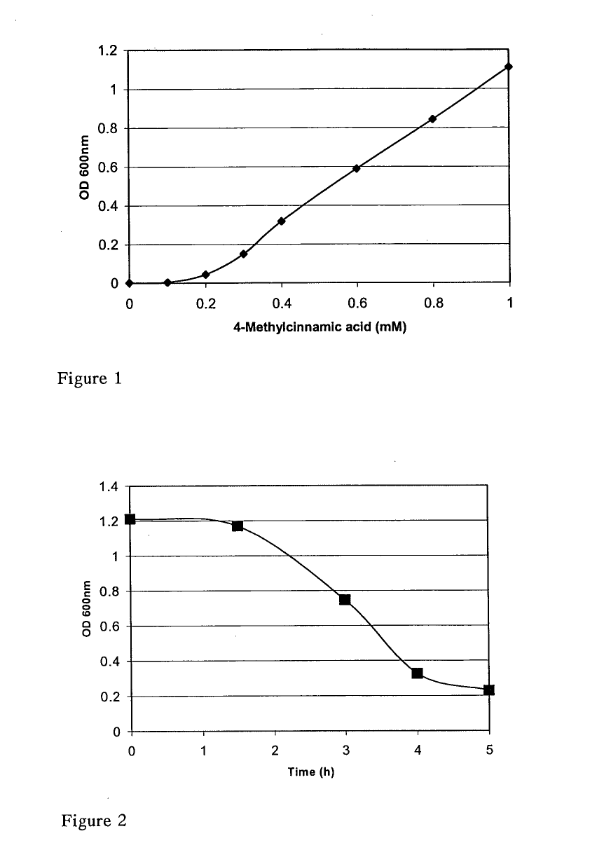

FIG. 1—is a calibration curve of the concentration of 4-methylcinnamic acid in ACM medium pH 4.0, detected by a spectrophotometer at 600 nm (OD 600nm);

FIG. 2—illustrates the disappearance of the dense cloud of 4-methylcinnamic acid after addition of A. niger spores. The strain of A. niger used is A. niger N402. 10⁶ A. niger conidiospores/ml were added to ACM medium (Aspergillus Complete Medium—which contains per one litre of water, 20 g glucose, 1.5 g casamino acids, 2 g bacteriological peptone, 1.5 g yeast extract, 6 g sodium nitrite, 0.52 g potassium chloride, 0.52 g magnesium sulphate heptahydrate, 1.52 g potassium orthophosphate, 1 mg ferrous sulphate, 1 mg zinc sulphate) at pH 4.0 containing 1 mM 4-methylcinnamic acid at time 0. Replicate cultures of 10 mls medium in 30 ml sealed bottles were incubated at 28° C. and shaken at 120 rpm on an orbital shaker. Each point represents the mean value of two bottles removed, sampled, and tested in a spectrophotometer at 600 nm;

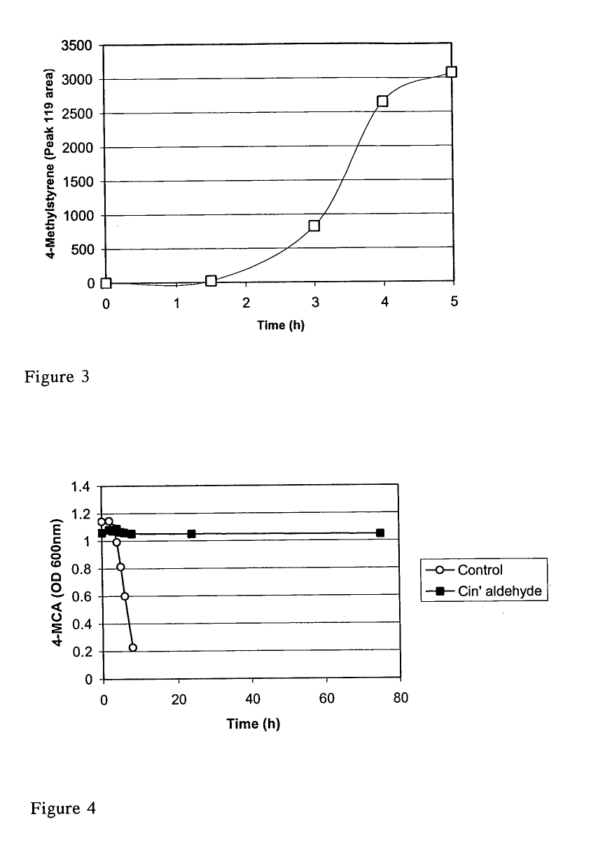

FIG. 3—illustrates the formation of 4-methylstyrene from 4-methylcinnamic acid after addition of A. niger spores. 10⁶ A. niger conidiospores/ml were added to ACM medium at pH 4.0 containing 1 mM 4-methylcinnamic acid at time 0. Replicate cultures of 10 mls medium in 30 ml sealed bottles were incubated at 28° C. and shaken at 120 rpm on an orbital shaker. Each point represents the mean value of two bottles removed, 0.2 mls headspace sampled, and tested by GCMS for 4-methylstyrene;

FIG. 4—illustrates that the presence of cinnamaldehyde inhibits the degradation of 4-methylcinnamic acid by A. niger spores. 10⁶ A. niger conidiospores/ml were added to ACM medium at pH 4.0 containing 1 mM 4-methylcinnamic acid at time 0 (control—circles). Replicate cultures contained 1 mM 4-methylcinnamic acid+1 mM cinnamaldehyde (squares). Bottles were incubated at 28° C. and shaken at 120 rpm on an orbital shaker.

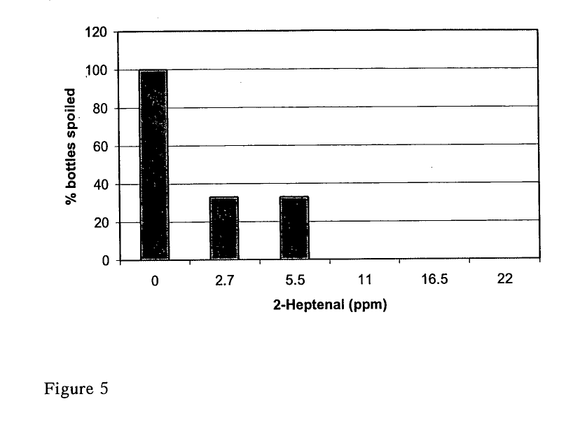

FIG. 5—illustrates the amount of 2-heptenal required to work in combination with 300 ppm of sorbic acid to prevent A. niger spoilage of a synthetic soft drink.

All chemicals used were supplied by Sigma/Aldrich and were of analytical grade.

A. niger conidiospore suspensions were obtained from 5 day old cultures of A. niger grown on agar slopes by scraping and vortexing spores into sterile water and 0.1% Tween. The number of spores in a spore suspension was counted using a microscope and haemocytometer slide.

### EXAMPLE 1

Assay

In order to demonstrate that compounds of Formula I can act as a preservative an assay system was devised. The compounds of Formula I are understood to work by acting to prevent or reduce the degradation of one or more additional preservatives in a food. For example, compounds of Formula I prevent the degradation of the preservative sorbic acid when conidiospores of certain moulds, such as A. niger, are present. In the absence of Formula I the sorbic acid would be decarboxylated, and its ability to act as a preservative against certain moulds reduced.

In the examples below a carboxylic acid similar to sorbic acid was used as it has unexpected properties which allow it to be easily assayed. The acid used was 4-methylcinnamic acid (4-MCA), which like sorbic acid is degraded by A. niger spores. Furthermore, tests show that spores of a strain of A. niger in which the padA1 gene had been disrupted did not degrade 4-MCA.

4-MCA is degraded by A. niger spores to produce 4-methyl stryrene as illustrated below.

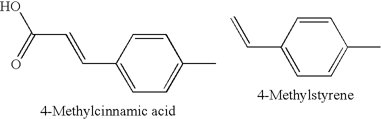

Unexpectedly 4-MCA forms a dense white cloud when added to water or growth media for moulds (such as ACM). The opacity of the cloud, when measured at an OD of 600 nm, is directly proportional to the concentration of 4-MCA. This makes it easy to assay for changes in 4-MCA levels. Up until now degradation of 4-MCA and sorbic acid has been determined through the detection of the volatile products produced after decarboxylation. The volatile products can be detected by using GCMS (Gas Chromatography/Mass Spectrometry), which is a slow and time consuming process.

### EXAMPLE 2

Degradation of 4-MCA by Germinating Mould Spores

To demonstrate that germinating conidiospores degrade 4-MCA, spores were added to a 1 mM solution of 4-MCA and degradation was monitored over time by checking the OD of the solution at 600 nm. FIG. 2 shows that over time nearly all the 4-MCA is degraded. It is understood that PadA1 in the germinating mould spores degrades the 4-MCA causing the white cloud to progressively disappear. Cloud disappearance is also accompanied by appearance of 4-methylstyrene in the headspace above the liquid medium (as illustrated in FIG. 3), which is measured by GCMS.

This example demonstrates that it is possible to use the disappearance of the 4-MCA cloud as a quick and convenient assay for PadA1 activity in mould spores.

### EXAMPLE 3

Inhibitors of 4-MCA Degradation

Unexpectedly, compounds according to Formula I have been found to inhibit, that is prevent or reduce, the degradation of 4-MCA by germinating conidiospores of A. niger (FIG. 4).

In particular, the aldhydes listed in Table 2 were tested and found to inhibit degradatiton of 4-MCA. More specifically it was observed that aldehydes with a double bond at position 2 and between 4 and 7 carbons in length inhibited 4-MCA degradation. Aldehydes unsaturated at positions 2 and 4 and between 6 and 9 carbons in length also inhibited 4-MCA breakdown. In addition, phenyl propiolic acid and phenyl propargyl aldehyde, with a triple bond at position 2, inhibited degradation.

All compounds were tested to see if they prevented the degradation of 4-MCA by adding 10⁶ A. niger conidiospores/ml to ACM medium at pH 4.0 containing 1 mM 4-methylcinnamic acid+1 mM putative inhibitor. Bottles were incubated at 28° C. and shaken at 120 rpm on an orbital shaker. Samples were taken at 5 and 72 hours.

TABLE 2

| Putative inhibitor | Alternative name | Use |
|---|---|---|
| 2-Butenal | Crotonaldehyde | |
| 2-Pentenal | | Flavour |
| 2-Hexenal | | Flavour |
| 2-Heptenal | | Flavour |
| 2,4-Hexadienal | Sorbic aldehyde | Flavour |
| 2,4-Heptadienal | | Flavour |
| 2,4-Octadienal | | Flavour |
| 2,4-Nonadienal | | Flavour |
| Cinnamaldehyde | | Flavour |
| phenyl propargyl | | |
| aldehyde | | |
| Phenyl propiolic acid | | |

The possibility that the compounds found to inhibit 4-MCA degradation were acting as toxic agents was examined, by determination of their MIC (Minimum Inhibitory Concentration) values. The MIC value is the lowest concentration of inhibitor that will completely prevent growth of a microbe. In this case, MIC values were determined by preparation of a triplicate series of bottles of ACM medium (pH 4.0 10 mls) each bottle containing progressively higher concentrations of an inhibitor. Bottles were inoculated with 1000 A. niger N402 conidiospores/ml and were incubated at 28° C. for 28 days. The MIC was the lowest concentration of inhibitor preventing mould growth in all three replicates, and any other higher concentration of inhibitor.

It was found that none of the compounds exert an antimicrobial effect that is sufficient at the concentrations used to explain the observed mould inhibition. Furthermore, other compounds generally more mould-toxic than the compounds listed in Table 2, were not able to inhibit mould growth when allied with 4-MCA at 1 mM e.g. (2,4-decadienal). 2,4-decadienal is more inhibitory and has a lower MIC than any of the compounds listed on Table 2, but it does not block PadA1 activity. Mould growth occurred in bottles containing 1 mM 4-MCA+1 mM 2,4-decadienal, but not in bottles containing 4-MCA +1 mM 2,4-heptadienal or other Table 2 compounds.

The data presented in Example 3 clearly demonstrate the ability of compounds of Formula 1 to enhance preservation by preventing the fungal degradation of the preservative.

### EXAMPLE 4

Amount of Compound of Formula I and Other Preservative Needed

Studies were undertaken to determine how much of a compound of Formula I was needed to protect another preservative, and in this example, another food preservative. Tests were carried out using ACM medium, pH 4.0, as a synthetic soft drink. Sorbic acid was added to the ACM, synthetic soft drink, at 300 ppm, which is the maximum level permitted in Europe.

The method used involved placing 10 ml of ACM pH 4.0+300 ppm sorbic acid (2.68 mM) in glass bottles (all experiments were carried out in triplicate) and inncoculating each 10 mls with 1000 conidiospores per ml of Aspergillus niger strain N402. Growth was measured after 28 days incubation at 28° C. PadA1 inhibitors of Formula I were added at 0, 0.025 mM, 0.05 mM, 0.1 mM, 0.15 mM, 0.2 mM, 0.3 mM, 0.4 mM, 0.5 mM, 0.6 mM, 0.8 mM, 1 mM.

The results show that 300 ppm sorbic acid was insufficient to prevent spoilage by A. niger conidiospores of the synthetic soft drink, but addition of the PadA1 inhibitor, or preservative protecting compound, prevented mould growth. Low levels of PadA1 inhibitors of Formula I were sufficient to protect the sorbic acid, e.g 11 ppm 2-heptenal was shown to be efficacious. FIG. 5 shows the effects of different amounts of 2-heptenal on the spoilage of the synthetic soft drink, indicated as a % bottles spoiled.

Table 3 shows the concentration of other compounds of Formula I that in combination with 300 ppm sorbic acid prevent spoilage of the synthetic soft drink.

TABLE 3

| | mM | ppm |
|---|---|---|
| Phenyl propiolic acid | 0.1 mM | 14.6 |
| Cinnamaldehyde | 0.15 mM | 20 |
| 2,4-Hexadienal | 0.05 mM | 5 |
| 2,4-Heptadienal | 0.05 mM | 5.5 |
| 2-Butenal | 0.1 mM | 7 |
| 2-Pentenal | 0.015 mM | 12 |
| 2-Hexenal | 0.05 mM | 5 |
| 2-Heptenal | 0.1 mM | 11 |
| 2-Octenal | 0.1 mM | 13 |
| 2-Nonenal | 0.1 mM | 14 |

1. Use of a compound of Formula I as a preservative: 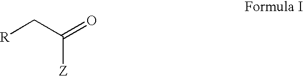
wherein:
the second carbon is optionally substituted;
the bond between R and the second carbon is unsaturated;
R is a C₁ to C₂₀ alkyl, aryl, alkaryl, alkenyl or alkynyl; and wherein R may be optionally substituted;
Z is H or OH; and
when Z is OH, the bond between R and the second carbon is a triple bond.

2. Use of a compound of Formula I to enhance the anti-mould efficacy of another preservative: 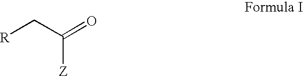
wherein:
the second carbon is optionally substituted;
the bond between R and the second carbon is unsaturated;
R is a Ci to C₂₀ alkyl, aryl, alkaryl, alkenyl or alkynyl; and wherein R may be optionally substituted;
Z is H or OH; and,
when Z is OH, the bond between R and the second carbon is a triple bond.

3. The use according to claim 2, wherein the other preservative is carboxylic acid.

4. A preservative composition or system comprising a first preservative and a compound of Formula I: 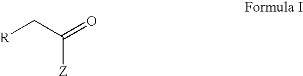
wherein:
the second carbon is optionally substituted;
the bond between R and the second carbon is unsaturated;
R is a C₁ to C₂₀ alkyl, aryl, alkaryl, alkenyl or alkynyl; and wherein R may be optionally substituted;
Z is H or OH; and
when Z is OH, the bond between R and the second carbon is a triple bond.

5. The preservative composition or system of claim 4, wherein the first preservative is a carboxylic acid.

6. Use of a composition comprising a carboxylic acid and a compound according to Formula I as a preservative: 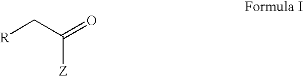
wherein:
the second carbon is optionally substituted;
the bond between R and the second carbon is unsaturated;
R is a Ci to C₂₀ alkyl, aryl, alkaryl, alkenyl or alkynyl; and wherein R may be optionally substituted;
Z is H or OH; and
when Z is OH, the bond between R and the second carbon is a triple bond.

7. A method of preserving a food comprising adding a compound of Formula I to the food: 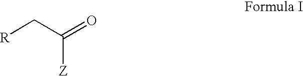
wherein:
the second carbon is optionally substituted;
the bond between R and the second carbon is unsaturated;
R is a Ci to C₂₀ alkyl, aryl, alkaryl, alkenyl or alkynyl; and wherein R may be optionally substituted;
Z is H or OH; and
when Z is OH, the bond between R and the second carbon is a triple bond.

8. The method according to claim 7, wherein a second preservative is added to the food.

9. The method according to claim 8, wherein the second preservative is a carboxylic acid.

10. A product comprising a compound of Formula I: 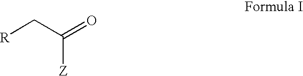
wherein:
the second carbon is optionally substituted;
the bond between R and the second carbon is unsaturated;
R is a C₁ to C₂₀ alkyl, aryl, alkaryl, alkenyl or alkynyl; and wherein R may be optionally substituted;
Z is H or OH; and
when Z is OH, the bond between R and the second carbon is a triple bond.

11. A product comprising a first preservative and a compound of Formula I: 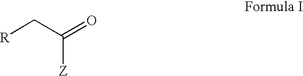
wherein:
the second carbon is optionally substituted;
the bond between R and the second carbon is unsaturated;
R is a Ci to C₂₀ alkyl, aryl, alkaryl, alkenyl or alkynyl; and wherein R may be optionally substituted;
Z is H or OH; and
when Z is OH, the bond between R and the second carbon is a triple bond.

12. The product of claim 11, wherein the first preservative is a carboxylic acid.

13. The use of claim 1, wherein Z is H.

14. The use of claim 1, wherein the compound has an R group which is a C₆ to C₉ alkyl, aryl, alkaryl, alkenyl or alkynyl and a double bond at the 2 and 4 positions.

15. The use of claim 1, wherein the compound of Formula I includes any of the group selected from 2-pentenal, 2-butenal, 2-hexenal, 2,4-hexadienal, 2-heptenal, 2,4-heptadienal, cinnamaldehyde, 2-octenal, 2,4-octadienal, 2-nonenal, 2,4-nonadienal, phenyl propiolic acid, phenyl propargyl aldehyde, α-amyl-cinnamaldehyde, 2-butyl-2-butenal, α-butyl cinnamaldehyde, citral (Neral, Geranial), 2-ethyl-2-heptenal, 2-furfurylidene butyraldehyde, α-hexyl-cinnamaldehyde, 2-isopropyl-5-methyl-2-hexenal, o-methoxy cinnamaldehyde, p-methoxy-α-methylcinnamaldehyde, 3-methyl-2-butenal, α-methylcinnamaldehyde, p-methylcinnamaldehyde, 2-methyl-3(2-furyl) acrolein, 2-methyl-2-octenal, 2-methyl-2-pentenal, 4-methyl-2-pentenal, 5-methyl-2-phenyl-2-hexenal, 4-methyl-2-phenyl-2-pentenal, 2-(methylthio)methyl-2-butenal, 2-methylthiomethyl-3-phenylpropenal, nona-2-trans, 6-cis, dienal, nona-2-trans, 6-trans, dienal, 2,6-octadienal, 2,4-pentadienal and 2-phenyl-2-butenal.
